(19) United States
(12) Patent Application Publication
(10) Pub. No.: US 2006/0280730 A1
(43) Pub. Date: Dec. 14, 2006

(54) INDUCTION OF APOPTOSIS OF MALIGNANT CELLS BY ACTIVATION OF CALCIUM-ACTIVATED POTASSIUM CHANNELS

(75) Inventors: Keith L. Black, Los Angeles, CA (US); Nagendra S. Ningaraj, Culver City, CA (US)

Correspondence Address: DAVIS WRIGHT TREMAINE LLP, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 11/466,049

(22) Filed: Aug. 21, 2006

Related U.S. Application Data
(62) Division of application No. 09/976,961, filed on Oct. 12, 2001.

(51) Int. Cl. A61K 38/46; A61K 31/13; A61K 38/22

(52) U.S. Cl. 424/94.6; 514/12; 514/663

(57) ABSTRACT

Disclosed are a method of inducing apoptosis of a malignant cell, which employs a calcium-activated potassium channel (K_{Ca}) activator and is useful for treating a malignant tumor in a human subject. Also disclosed are methods of selectively inhibiting the proliferation of malignant cells compared to non-malignant cells in a mixed population of malignant and non-malignant cells and of inhibiting the growth of a malignant tumor, such as a glial tumor, in a mammalian subject. A kit for inducing apoptosis of malignant cells is also disclosed.

### BACKGROUND OF THE INVENTION

Throughout the application various publications are referenced in parentheses. The disclosures of these publications in their entireties are hereby incorporated by reference in the application in order to more fully describe the state of the art to which this invention pertains.

1. The Field of the Invention

This invention relates to the medical arts. In particular, it relates to a method of selectively inducing death of malignant cells in vitro and in vivo.

2. Discussion of the Related Art

There are four main types of potassium channels: inverse rectifier potassium channels (Kᵢᵣ); voltage-gated potassium channels (Kᵥ); calcium-activated potassium channels (Ca²⁺-activated K⁺ channel; i.e., K_{Ca}); and ATP-sensitive potassium channels (K_{ATP}). (Nelson, M. T. and Quayle, J. M., Physiological roles and properties of potassium channels in arterial smooth muscle, Am. J. Physiol. 268(4 Pt 1):C799-822 [1995]). The K_{Ca} and K_{ATP} potassium channels are ubiquitously distributed in tissues including brain capillaries. The K_{Ca} is an important regulator of cerebral blood vessel tone (Nelson M T, Quayle J M. Physiological roles and properties of potassium channels in arterial smooth muscle, Am. J. Physiol. 268(4 Pt 1): C799-822[1995]). The K_{Ca} channel is ubiquitously distributed in tissues as α and β subunits. Its activity is triggered by depolarization and enhanced by an increase in cytosolic calcium dication (Ca²⁺). A local increase in Ca²⁺ is sensed by the extremely sensitive brain α-subunit of the K_{Ca}, directed towards the cytoplasm in the cell, that allows a significant potassium cation flux through these channels.

There is growing evidence that membrane ion channels are involved in cell differentiation and proliferation. Potassium channels interfere with a variety of different cell lines derived from breast carcinoma (Wegman et al. Pfuegers Arch. 417:562-570 [1991]), melanoma (Wienhuesal et al. 151:149-157 [1996]), and neuroblastoma (Dubois B and Dubois J M. Cellular Signaling 4:333-339 [1991]). The K_{Ca} channels are known to regulate cell membrane potential and, thus, may have a role in cell proliferation. Biochemical modulation of K_{Ca} channels induces K⁺ flux causing membrane hyperpolarization affecting the entry of calcium dication. Excessive K⁺ conductance causes reduction in membrane potential, induces cell death by apoptosis or necrosis in hypoxia and ischemia. Brain tumor cells appear to express immunopositive K_{Ca} channels as studied immunohistochemically with polyclonal anti-K_{Ca} antibody. Further, others have shown that K_{Ca} channels expressed on human glioma cells are highly sensitive to [Ca²⁺]ᵢ concentration. However, the effect of K_{Ca} channel activation in glioma cell proliferation has not been so far studied.

Treatments directed to the use of potassium channel activators or agonists have been taught for disorders including hypertension, cardiac and cerebral ischemia, nicotine addiction, bronchial constriction, and neurodegenerative diseases, and for increasing the permeability of the blood brain barrier. (Erhardt et al., Potassium channel activators/openers, U.S. Pat. No. 5,416,097; Schohe-Loop et al., 4,4′-bridged bis-2,4-diaminoquinazolines, U.S. Pat. No. 5,760,230; Sit et al., 4-aryl-3-hydroxyquinolin-2-one derivatives as ion channel modulators, U.S. Pat. No. 5,922,735; Garcia et al., Biologically active compounds, U.S. Pat. No. 5,399,587; Cherksey, Potassium channel activating compounds and methods of use thereof, U.S. Pat. No. 5,234,947).

Apoptosis is programmed cell death, as signaled by the nuclei in normally functioning human and animal cells, when age or state of cell health and condition dictates. Apoptosis is an active process requiring metabolic activity by the dying cell, often characterized by cleavage of the DNA into fragments that give a so called laddering pattern on gels. Cancerous cells, however, are typically unable to experience the normal cell transduction or apoptosis-driven natural cell death process. Consequently, mechanisms have been sought by which apoptosis may be induced in malignant cells.

Mechanisms of induction of apoptosis in several different cell types and under various physiological conditions have been at least partially studied, and some apoptotic mechanisms appear to be mediated by complex signal transduction pathways involving the phosphorylation and/or dephosphorylation of signal transducing peptides. For example, phosphotyrosine phosphatase inhibitors or activators of protein tyrosine kinase induced apoptosis in B- and T-lymphocytes. (Schieven, G. L., Phosphotyrosine phosphatase inhibitors or phosphotyrosine kinase activators for controlling cellular proliferation, U.S. Pat. No. 5,877,210; Schieven, G. L., Use of phosphotyrosine phosphatase inhibitors for controlling cellular proliferation, U.S. Pat. No. 5,693,627). Also, expression of cytoplasmic Bruton's tyrosine kinase (BTK) has been linked to apoptosis in some cell lines. (Islam, T. C. et al., BTK mediated apoptosis, a possible mechanism for failure to generate high titer retroviral producer clones, J. Gene Med. 2(3):204-9 [2000]). On the other hand, signaling by activated Signal Transducers and Activators of Transcription (STATs) may participate in oncogenesis by stimulating cell proliferation and preventing apoptosis. (E.g., Bowman, T. et al., STATs in oncogenesis, Oncogene 19(21):2474-88[2000]; Reddy, E. P., et al., IL-3 signaling and the role of Src kinases, JAKs and STATs: covert liason unveiled, Oncogene 19(21):2532-47 [2000]).

Some hypothetical apoptotic mechanisms may be mediated by the activity of certain varieties of potassium channel, but contrary and varied effects indicate that different potassium channels might play different and specific mechanistic roles in apoptosis, if they play any direct role at all. For example, the Kᵥ1.3 voltage-gated potassium channel has been implicated in the pathway for Fas-induced apoptosis. (E.g., Gulbins, E. et al., Ceramide-induced inhibition of T-lymphocyte voltage-gated potassium channel is mediated by tyrosine kinases, Proc. Natl. Acad. Sci. USA 94(14):7661-6 [1997]). Expression of Kᵢᵣ1.1 potassium channel from an expression vector caused apoptosis in hippocampal neurons. (Nadeau, H. et al., ROMK1 (Kᵢᵣ1.1) cause cipoptosis and chronic silencing of hippocampal neurons, J. Neurophysiol. 84(2):1062-75 [2000]). Also, tumor necrosis factor (TNF)-α-mediated apoptosis of liver cells was dependent on activation of unspecified potassium channels and chloride channels and was further dependent on the presence of calcium dication and protein kinase C activity. (Nietsch, H. H. et al., Activation of potassium and chloride channels by tumor necrosis factor alpha, J. Biol. Chem. 275(27):20556-61 [2000]).

In contrast, the K_{ATP} potassium channel activator cromakalim prevented glutamate-induced or glucose/hypoxia-induced apoptosis in hippocampal neurons. (Lauritzen, I. et al., The potassium channel opener (−)-cromakalim prevents glutamate-induced cell death in hippocampal neurons, J Neurochem, 69(4):1570-9 [1997]). Clofilium an inhibitor of the Kᵥ1.5 delayed rectifier potassium channel, induced apoptosis of human promelocytic leukemia (HL-60) cells. (Choi, B. Y. et al., Clofilium, a potassium channel blocker, induces apoptosis of human promelocytic leukemia (HL-60) cells via Bcl-2-insensitive activation of caspase-3, Cancer Lett, 147 (1-2):85-93 [1999]; Malayev, A. A. et al., Mechanism of clofilium block of the human Kv1.5 delayed rectifier potassium channel, Mol. Pharmacol. 47(1):198-205 [1995]). Also, Kᵥ inhibitor 4-aminopyridine induced apoptosis in HepG2 human hepatoblastoma cells. (Kim, J. A. et al., Ca²⁺ influx mediates apoptosis induced by 4-aminopyridine, a K⁺ channel blocker, HepG2 human hepatoblastoma cells, Pharmacology 60(2):74-81 [2000]).

Thus, links between various types of potassium channels and any particular mechanisms of apoptosis remain unclear, and no role for calcium-activated potassium channels (K_{Ca}), in particular, has been suggested.

The ability to induce apoptosis in malignant cells would be especially desirable with respect to malignant tumors, especially tumors of the central nervous system. These malignancies are usually fatal, despite recent advances in the areas of neurosurgical techniques, chemotherapy and radiotherapy.

The glial tumors, or gliomas, comprise the majority of primary malignant brain tumors. Gliomas are commonly classified into four clinical grades, with the most aggressive or malignant form of glioma being glioblastoma multiforme (GBM; also known as astrocytoma grade IV), which usually kills the patient within 6-12 months. (Holland, E. C. et al., Combined activation of Ras and Akt in neural progenitors induces glioblastoma formation in mice, Nat. Genet. 25(1):55-57 [2000]; Tysnes, B. B et al., Laminin expression by glial fibrillary acidic protein positive cells in human gliomas, Int. J. Dev. Neurosci. 17(5-6):531-39 [1999]).

GBM tumors are characterized by rapid cell growth and extensive invasion into the surrounding normal brain tissue. GBM tumors are difficult to remove surgically and typically recur locally at the site of resection, although metastases also may occur within the central nervous system. Tumor cell movement within the central nervous system is a complex process that involves tumor cell attachment to the extracellular matrix (ECM) via cell surface receptors, degradation of the ECM by proteolytic enzymes, including serine proteases and matrix metalloproteinases, and subsequent tumor cell locomotion. (Tysnes et al. [1999]; MacDonald, T. J. et al., Urokinase induces receptor mediated brain tumor cell migration and invasion, J. Neurooncol. 40(3):215-26 [1998]; Mäenpää, A. et al., Lymphocyte adhesion molecule ligands and extracellular matrix proteins in gliomas and normal brain: expression of VCAM-1 in gliomas, Acta Neuropathol. (Berl.) 94(3):216-25 [1997]). Thus, malignant gliomas overexpress members of the plasminogen activator system and characteristically invade by migrating on ECM-producing white matter tracts and blood vessel walls. (Tysnes et al. [1999]; Colognato, H. and Yurchenco, P. D., Form and function: the laminin family of heterotrimers, Dev. Dyn. 218(2):213-34 [2000]).

Despite a wealth of molecular biological, biochemical and morphological information that is available today on gliomas, the prognosis with treatment has not significantly changed in the last two decades and remains among the worst for any kind of malignancy. (E.g., Shapiro, W. R., Shapiro, J. R., Biology and treatment of malignant glioma, Oncology 12:233-40 [1998]; Thapar, K. et al., Neurogenetics and the molecular biology of human brain tumors, In: Brain Tumors, Edit. Kaye A H, Laws E R, pp. 990. [1997]). In particular, there are no standard therapeutic modalities that can substantially alter the prognosis for patients with malignant glial tumors of the brain, cranium, and spinal cord. Although intracranial tumor masses can be debulked surgically, treated with palliative radiation therapy and chemotherapy, the survival associated with intracranial glial tumors, for example, a glioblastoma, is typically measured in months.

The present invention provides a much needed method of inducing apoptosis in glioma cells, in vitro and in vivo, that employs activators of calcium-activated potassium channels This and other benefits of the present invention are described herein.

### SUMMARY OF THE INVENTION

The disclosed invention is directed to a method of inducing apoptosis in a malignant cell, such as a glioma cell. The method involves treating the malignant cell with a calcium-activated potassium channel (Ca²⁺-activated K⁺ channel; i.e., K_{Ca}) activator, such as, but not limited to NS-1619, which is administered under conditions and in an amount sufficient to induce apoptosis of the cell, i.e., programmed cell death. In contrast, normal cells, such as normal human brain endothelial cells and human fetal astrocytes, are insensitive to the K_{Ca} activator and are not adversely affected by the treatment. Hence the present invention relates to a method of selectively inhibiting the proliferation of malignant cells compared to non-malignant cells in a mixed population of malignant and non-malignant cells, whether in vitro or in vivo. The method involves administering to the mixed population of malignant and non-malignant cells a calcium-activated potassium channel activator in an amount sufficient to induce apoptosis of at least a plurality of malignant cells compared to non-malignant cells, thereby selectively inhibiting the proliferation of malignant cells.

Since the present invention is capable of selectively targeting malignant cells, whether in vitro or in vivo, the present invention also relates to a method of inhibiting the growth of a malignant tumor, such as a glial tumor, in a mammalian subject. The method involves administering to a mammalian subject having a malignant tumor, which comprises a malignant cell, a calcium-activated potassium channel activator under conditions and in an amount sufficient to induce apoptosis of the cell, thereby inhibiting growth of the tumor.

Thus, the invention provides a useful addition to the pharmaceutical anti-cancer armamentarium, especially for treating patients who do not respond well to commonly used chemotherapeutic agents. Moreover, the administration of K_{Ca} activators is not associated with the debilitating systemic side effects typical of the cytotoxic agents currently used in anti-cancer chemotherapy.

Useful kits are also provided for facilitating the practice of the inventive methods.

These and other advantages and features of the present invention will be described more fully in a detailed description of the preferred embodiments which follows.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows in vitro effect of K_{Ca} activator NS-1619 on in vitro cell proliferation, as measured by optical density (OD) at 450 nm wavelength, by rat glioma cells (RG2, C6, and 9L). **=P<0.01; ***=P<0.001.

FIG. 2 shows in vitro effect of K_{Ca} activator NS-1619 on in vitro cell proliferation, as measured by optical density (OD) at 450 nm wavelength, by primary human glioma cell lines (GBM and astrocytoma) or human normal cell lines (brain microvessel endothelial cells [HBMVEC], or fetal astrocytes [HFA]). **=P<0.01; ***=P<0.001.

FIG. 3 illustrates a differential sensitivity to NS-1619 (50 μg/mL) that was observed among rat glioma cell lines RG2, C6, and 9L.

FIG. 4 shows a differential sensitivity to NS-1619 at 50 μg/mL among primary human GBM, astrocytoma, and U373MG glioma cells. Relative insensitivity of normal cell lines HBMVEC and HFA is also shown.

FIG. 5 illustrates the in vitro effect of K_{Ca} activator NS-1619 on in vitro U87 cell proliferation, as measured by apoptosis assay and FACS analysis. FIG. 5A shows the effect of vehicle (negative control); FIG. 5B shows the effect on U87 cells of 50 μg/mL NS-1619; and FIG. 5C shows the effect of 5 μM staurosporin (positive control).

FIG. 6 illustrates FACS detection of apoptosis induction by NS-1619 (50 μg/mL) in malignant rat cells in vitro, compared to untreated controls: RG2 cells (NS-1619-treated [FIG. 6A] and control [FIG. 6B]); C6 cells (NS-1619-treated [FIG. 6C] and control [FIG. 6D]); and 9L cells (NS-1619-treated [FIG. 6E] and control [FIG. 6F]). SSC-Height=size scattering height, which indicates degree of granularity or complexity of cells; FSC-Height=forward scattering height, which indicates size of events or cells; PI=magnitude of propidium iodide staining; annexin V-FITC=magnitude of annexin V-fluorescein isothiocyanate staining.

FIG. 7 illustrates FACS detection of apoptosis induction by NS-1619 (50 μg/mL) in malignant human cells in vitro (GBM primary cell line), compared to untreated controls, and normal (i.e., non-malignant) human cells (HBMVEC): GBM cells (NS-1619-treated [FIG. 7A] and control [FIG. 7B]); HBMVEC cells (NS-1619-treated [FIG. 7C] and control [FIG. 7D]). SSC-Height=size scattering height, which indicates degree of granularity or complexity of cells; FSC-Height=forward scattering height, which indicates size of the event; PI=magnitude of propidium iodide staining; annexin V-FITC=magnitude of annexin V-fluorescein isothiocyanate staining.

FIG. 8 illustrates FACS detection of apoptosis induction by intracarotid (100 μg NS-1619/kg/min for 15 minutes, flow rate=0.0823 mL/min) injection of NS-1619 in Wistar rats having implanted RG2 gliomas, in vivo, compared to untreated controls: glioma tumor after 2 daily infusions of vehicle ([3.2 mL PBS+0.5% ethanol]/kg body mass/day; FIG. 8A); contralateral normal brain tissue after vehicle infusion on 2 days (FIG. 8B); RG2 glioma tissue after 2 daily infusions of vehicle containing different doses of NS-1619 (50 μg NS-1619/kg body mass/day; FIG. 8C) and normal contralateral brain tissue of the same rat treated with NS-1619 (FIG. 8D); (100 μg NS-1619/kg/day; FIG. 5E) and normal contralateral brain tissue of the same rat (FIG. 8F); 100 μg NS-1619/kg/day; FIG. 5G) and normal contralateral brain tissue of the same rat (FIG. 8H); FIG. 8I shows as a positive control the apoptotic effect of staurosporin (intracarotid injection total 20 μg/kg body mass, flow rate=0.0823 mL/min, for 15 minutes) on RG2 tumor cells implanted in Wistar rats, and FIG. 8J shows the effect on contralateral tissue. SSC-Height=size scattering height, which indicates degree of granularity or complexity of cells; FSC-Height=forward scattering height, which indicates size of the event; FL1-Height is a measure of FITC-Annexin V staining that indicates apoptotic cells; FL2-Height is a measure of propidium iodide (PI) staining that indicates necrotic cells.

FIG. 9 illustrates the in vivo apoptosis-inducing effect, in Wistar rats having implanted RG2 tumors, of intracarotid doses of NS-1619 on three consecutive days. RG2 tumor after 3 daily infusions of vehicle ([3.2 mL PBS+1% ethanol]/kg body mass/day, flow rate=0.0823 mL/min, for 15 minutes; FIG. 9A); contralateral normal brain tissue after vehicle infusion on 3 days (FIG. 9B). FIG. 9C shows the result of three consecutive daily treatments with NS-1619 (100 μg/kg body mass/day; dose flow rate=0.0823 mL/min, for 15 minutes) in implanted RG2 glioma tumors, compared with contralateral normal brain (FIG. 9D). SSC-Height=size scattering height, which indicates degree of granularity or complexity of cells; FSC-Height=forward scattering height, which indicates size of the event; FL1-Height is a measure of FITC-Annexin V staining that indicates apoptotic cells; FL2-Height is a measure of propidium iodide (PI) staining that indicates necrotic cells.

FIG. 10 shows that valinomycin (a K⁺ ionophore) inhibited PTK activity in RG2 cell lysate in a dose-dependent manner, while NS-1619 did not significantly affect PTK activity in RG2 cell lysate. Values are mean±SD of six experiments.

FIG. 11 shows intense over-expression of K_{Ca} as indicated by anti-K_{Ca} immunostain of rat glioma tissue (FIG. 11B), compared to normal contralateral brain tissue (FIG. 11A). Magnification is 100×.

### DETAILED DESCRIPTION OF THE PREFERRED EMBODIMENTS

The present invention relates to a method of inducing apoptosis in malignant cells, such as glioma cells, whether in vitro or in vivo. Thus, the present invention also relates to a method of inhibiting the growth of a malignant tumor in a mammalian subject.

A malignant tumor includes, but is not limited to, a glioma, a glioblastoma, an oligodendroglioma, an astrocytoma, an ependymoma, a primitive neuroectodermal tumor, an atypical meningioma, a malignant meningioma, a neuroblastoma, a sarcoma, a melanoma, a lymphoma, or a carcinoma. The malignant tumor can be contained within in the skull, brain, spine, thorax, lung, abdomen, peritoneum, prostate, ovary, uterus, breast, stomach, liver, bowel, colon, rectum, bone, lymphatic system, or skin, of the mammalian subject.

Among malignant tumors for which the inventive methods are effective are gliomas, which include any malignant glial tumor, i.e., a tumor derived from a transformed glial cell. A glial cell includes a cell that has one or more glial-specific features, associated with a glial cell type, including a morphological, physiological and/or immunological feature specific to a glial cell (e.g. astrocyte or oligodendrocyte), for example, expression of the astroglial marker fibrillary acidic protein (GFAP) or the oligodendroglial marker O4. Gliomas include, but are not limited to, astrocytoma grade II, anaplastic astrocytoma grade III, astrocytoma with oligodendrogliomal component, oligodendroglioma, and glioblastoma multiforme (GBM; i.e., astrocytoma grade IV).

The inventive methods are useful in treating malignant cells originating from, or found in, any mammal, including a human, non-human primate, canine, feline, bovine, porcine or ovine mammal, as well as in a small mammal such as a mouse, rat, gerbil, hamster, or rabbit.

The method of inducing apoptosis in a glioma cell involves treating the cell with a calcium-activated potassium channel (K_{Ca}) activator, under conditions and in an amount sufficient to induce apoptosis of the cell, i.e., programmed cell death.

Examples of useful K_{Ca} activators include 1,3-dihydro-1-[2-hydroxy-5-(trifluoromethyl)phenyl]-5-(trifluoromethyl)-2H-benzimidazol-2-one (NS-1619, a specific large conductance K_{Ca} activator; RBI, Natick, Mass.), or 1-ethyl-2-benzimidazolinone (1-EBIO). Also included among useful K_{Ca} activators is the vasodilator bradykinin (Arg-Pro-Pro-Gly-Phe-Ser-Pro-Phe-Arg), or a polypeptide bradykinin analog, such as receptor mediated permeabilizer (RMP)-7 or A7 (e.g., Kozarich et al., U.S. Pat. No. 5,268,164 and PCT Application No. WO 92/18529). Other useful analogs of bradykinin include related peptide structures which exhibit the same properties as bradykinin but have modified amino acids or peptide extensions on either terminal end of the peptide. For example, such bradykinin analogs include [phe.sup.8(CH.sub.2-NH)Arg.sup.9]-bradykinin, N-acetyl [phe.sup.8(CH.sub.2-NH-Arg.sup.9]bradykinin and desArg9-bradykinin. For the purposes of the present invention, other useful K_{Ca} activators include soluble guanylyl cyclase activators, such as, metalloporphyrins (e.g., zinc or tin protoporphyrin IX), YC-1 (a benzyl indazole derivative), or guanylyl cyclase activating proteins (GCAPs).

Included among useful K_{Ca} channel activators are pharmaceutically acceptable molecular conjugates or salt forms that still have activity as K_{Ca} channel activators. Examples of pharmaceutically acceptable salts comprise anions including sulfate, carbonate, bicarbonate, nitrate, or the like. Other embodiments of pharmaceutically acceptable salts contain cations, such as sodium, potassium, magnesium, calcium, or the like. Other embodiments of useful potassium channel agonists are hydrochloride salts.

In accordance with the inventive method, “administering” the K_{Ca} channel activator to a cell, or population of cells, includes exposing the cell to the activator, applying the activator to the cell, giving the activator to the cell, treating or bathing the cell with the activator, or, particularly for in vitro embodiments, adding the activator to a liquid, semi-solid, or solid cell culture medium containing or supporting the cell. Preferably, quantities of a channel activator sufficient to induce apoptosis of the malignant cell, in vitro, including under normal physiological conditions (e.g., normal physiological pH, oxygenation, and nutrient repletion), generally range from about 5 μg/mL to about 100 μg/mL, and more preferably about 50 μg/mL to about 100 μg/mL, but sufficient quantities appropriate for particular malignant cell types are readily determined by routine in vitro screening methods.

In accordance with the inventive method, applied in vivo, the calcium-activated potassium channel activator is preferably administered to the mammalian subject by a transvascular delivery route, for example, by intravenous or intra-arterial injection or infusion. For treating an intracranial tumor, the calcium-activated potassium channel activator is preferably administered to the mammalian subject by intracarotid infusion.

In other preferred in vivo embodiments, administration of the K_{Ca} activator to the mammalian subject, for delivery to a malignant tumor, is by intratumoral injection through a surgical incision, for example, through a craniotomy for a brain tumor. Typically, but not necessarily, surgical debulking of the tumor is done, if possible, before injection of the K_{Ca} activator into the remaining tumor mass containing malignant cells. Also for treating a brain tumor, another preferred delivery method is stereotactic injection of the K_{Ca} activator into the malignant tumor at a site having pre-established coordinates.

For in vivo embodiments of the inventive methods, the amount of K_{Ca} activator to be administered ranges from 0.075 to 1500 micrograms per kilogram body mass. For humans the range of 0.075 to 150 micrograms per kilogram body mass is most preferred. This can be administered in a bolus injection, but is preferably administered by infusion over a period of one to thirty minutes, and most preferably during a period of one to about fifteen minutes. For example, in rats, a dose rate of about 0.75 to about 100 μg kg⁻¹ min⁻¹ is most suitable. At dose rates above about 200 μg kg⁻¹ min⁻¹ a concomitant fall in blood pressure has been observed In humans, effective dose rates are about 0.075 to about 15 μg kg⁻¹ min⁻¹, with cautious monitoring of blood pressure being advised. The practitioner skilled in the art is also cautious in regulating the total infusion volume, rate of liquid infusion, and electrolyte balance to avoid adverse physiological effects related to these.

For example, for delivery by intravascular infusion or bolus injection into a mammal, such as a human, the K_{Ca} activator is preferably in a solution that is suitably balanced, osmotically (e.g., about 0.15 M saline) and with respect to pH, typically between pH 7.2 and 7.5; preferably the solution further comprises a buffer, such as a phosphate buffer (e.g., in a phosphate buffered saline solution). The solution is formulated to deliver a dose of about 0.075 to 1500 micrograms of K_{Ca} activator per kilogram body mass in a pharmaceutically acceptable fluid volume over a maximum of about thirty minutes. For human subjects, the solution is preferably formulated to deliver a dose rate of about 0.075 to 150 micrograms of potassium channel agonist per kilogram body mass in a pharmaceutically acceptable fluid volume over a period of up to about thirty minutes.

In accordance with the inventive method, adminstration of the K_{Ca} activator is preferably, but not necessarily, repeated, as described hereinabove, for two to three consecutive days.

Some useful K_{Ca} channel activators, such as NS-1619, are not easily dissolved in water; in preparing these agents for administration, a suitable and pharmaceutically acceptable solvent, such as ethanol (e.g., 25% v/v ethanol or higher ethanol concentrations), can be used to dissolve the K_{Ca} potassium channel activator, prior to further dilution with an infusion buffer, such as PBS. The skilled practitioner is cautious in regulating the final concentration of solvent in the infusion solution to avoid solvent-related toxicity. For example, a final ethanol concentration in an infusion solution up to 5-10% (v/v) is tolerated by most mammalian subjects with negligible toxicity.

While the inventive method does not depend on any particular mechanism or signal transduction pathway by which apoptosis is induced, it is thought that administration of the potassium channel activator increases potassium flux through calcium-activated potassium channels in the cell membranes of malignant cells and in endothelial cell membranes of the capillaries and arterioles delivering blood to malignant tumors. In practicing the inventive methods, it is not necessary to measure potassium channel activity (i.e., potassium cation flux therethrough). But the skilled artisan is aware that potassium flux can be measured by any suitable method, for example, by measuring cellular uptake of ⁴²K⁺ or ²⁰¹Tl⁺ or channel conductance using patch-clamp or microelectrode devices. (e.g., T. Brismar et al., Thallium-201 uptake relates to membrane potential and potassium permeability in human glioma cells, Brain Res. 500(1-2):30-36 [1989]; T. Brismar et al., Mechanism of high K⁺ and Tl⁺ uptake in cultured human glioma cells, Cell Mol. Neurobiol. 15(3):351-60 [1995]; S. Cai et al., Single-channel characterization of the pharmacological properties of the K(Ca2+) channel of intermediate conductance in bovine aortic endothelial cells, J. Membr. Biol. 163(2):147-58 [1998]).

The invention also relates to a kit for inducing apoptosis in a malignant cell in accordance with the inventive methods described herein. The kit is an assemblage of materials or components, including a K_{Ca} potassium channel activator in a pharmaceutically acceptable formulation, as described above. In addition, the kit contains instructions for using the K_{Ca} activator in accordance with the inventive methods. Optionally, the kit also contains other components, such as, diluents, buffers, pharmaceutically acceptable carriers, pipetting or measuring tools or paraphernalia for injection or infusion, for example syringes, stents, catheters, infusion lines, clamps, and/or infusion bags/bottles, which can contain a pharmaceutically acceptable formulation of the K_{Ca} activator. The pharmaceutically acceptable formulation contains the K_{Ca} activator and can also optionally contain one or more pharmaceutically acceptable carrier(s). As used herein, the term “acceptable carrier” encompasses any of the standard pharmaceutical carriers. The carrier can be an organic or inorganic carrier or excipient, such as water and emulsions such as an oil/water or water/oil emulsion, and various types of wetting agents. The active ingredient(s) can optionally be compounded in a composition formulated, for example, with non-toxic, pharmaceutically acceptable carriers for injections, infusions, tablets, pellets, capsules, solutions, emulsions, suspensions, and any other form suitable for use. Such carriers also include glucose, lactose, gum acacia, gelatin, mannitol, starch paste, magnesium trisilicate, talc, corn starch, keratin, colloidal silica, potato starch, urea, medium chain length triglycerides, dextrans, normal saline, phosphate buffered saline and other carriers suitable for use in manufacturing preparations, in solid, semisolid, or liquid form. In addition auxiliary, stabilizing, thickening and coloring agents and perfumes can be used as appropriate.

Optionally, the kit also contains other useful components. The materials or components assembled in the kit can be provided to the practitioner stored in any convenient and suitable ways that preserve their operability and utility. For example the components can be dissolved, dehydrated, or lyophilized form; they can be provided at room, refrigerated or frozen temperatures.

The exact nature of the components configured in the inventive kit depends on its intended purpose. For example, some embodiments of the kit are configured for the purpose of treating cultured mammalian cells. Other embodiments are configured for the purpose of treating mammalian cells in vivo, i.e., for treating mammalian subjects in need of treatment, for example, subjects with malignant tumors. Preferred embodiments are directed to treating gliomas. In a most preferred embodiment, the kit is configured particularly for the purpose of treating human subjects.

Instructions for use are included in the kit. “Instructions for use” typically include a tangible expression describing the reagent concentration or at least one assay method parameter, such as the relative amounts of reagent and sample to be admixed, maintenance time periods for reagent sample admixtures, temperature, buffer conditions, and the like, typically for an intended purpose.

The components are typically contained in suitable packaging material(s). As employed herein, the phrase “packaging material” refers to one or more physical structures used to house the contents of the kit, such as, the K_{Ca} activator. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass or plastic vial or ampoule. The packaging material generally has an external label which indicates the contents and/or purpose of the kit and/or its components.

The foregoing descriptions of the methods and kits of the present invention are illustrative and by no means exhaustive. The invention will now be described in greater detail by reference to the following non-limiting examples.

### EXAMPLES

### Example 1

### Methods

Cells. Primary cell lines were prepared from human gliomas (glioblastoma multiforme [GBM] or astrocytoma) or established rat glioma cell lines (RG2, C6 and 9L) were used. Cultured human normal cell lines, brain microvessel endothelial cells (HBMVEC), or fetal astrocytes were used.

Cell proliferation assay. Cells (5×10⁴ cells) were cultured in each well of 96-well microtiter culture plates and were allowed to achieve confluency to form a monolayer of cells in each well. For dose response studies cells were incubated with different concentrations (1-100 μg/mL) of NS-1619 or minoxidil sulfate for 4 hours at 37° C. in a CO₂ (5%) incubator. Cells washed twice carefully with respective medium, and allowed to incubate overnight at 37° C. under 5% CO₂. The following day, cells were incubated with WST-1 reagent (Boehringer Mannheim) at 37° C. in a CO₂ (5%) incubator for 60-90 minutes, and optical density at 450 nm was measured with a 96-well plate reader. The magnitude of OD indicated the number of viable cells, greater the OD, higher the number of viable cells, correlated a standard curve of cell numbers.

In vitro apoptosis studies in human cells. Human glioma cells (U-87) and HBMVECells were seeded into a 25-mL tissue-culture flask. After 24 h (about 60-70% confluencey), cells were treated with either NS-169 (50 or 100 micrograms/mL) or staurosporine (positive control; 5 μM), a known inducer of apoptosis (Tamaoki, T., et al., Staurosporine, a potent inhibitor of phospholipid/Ca⁻⁺ dependent protein kinase, Biochem. Biophys. Res. Commun. 135: 397-402 [1986]; Matsumoto, H., and Sasaki, Y., Staurosporine, a protein kinase C inhibitor interferes with proliferation of arterial smooth muscle cells, Biochem. Biophys. Res. Commun. 158:105-109 [1989]). U-87 cells were returned to the CO₂ incubator and incubated overnight. The following day, cells were washed twice with fresh growth medium and cells prepared for FACS analysis as described hereinbelow.

Apoptosis Assay. The changes in plasma membrane are one of the earliest events in cell death. In apoptotic cells the membrane phospholipid phosphatidylserine (PS) is translocated from the inner to the outer leaflet of the cell membrane in the early phases of apoptosis. Annexin V is a Ca²⁺-dependent, phospholipid-binding protein that binds to PS with high affinity. Consequently, Annexin V (PharMingen) conjugated to a label, such as fluorescein isothiocyanate (FITC) or biotin, serves as a sensitive probe for flow cytometry analysis of cells that are undergoing apoptosis. The manufacturers suggested protocol was followed. (PharMingen; Vermes, I. et al., A novel assay for apoptosis. Flow cytometric detection of phosphatidylserine expression on early apoptotic cells using fluorescein labelled Annexin V, J. Immunol. Meth. 184:39-51 [1995]). Concurrently, propidium iodide is typically used as a standard flow cytometric viability probe, because the intact cell membranes of viable cells exclude propidium iodide, whereas the cell membranes of dead and damaged cells are permeable to propidium iodide. Cells that stain positive for Annexin V and negative for propidium iodide are undergoing apoptosis; cells that stain positive for both Annexin V and propidium iodide are either at the end stage of apoptosis, are undergoing necrosis, or are already dead; and cells that stain negative for both Annexin V-biotin and propidium iodide are alive and not undergoing measurable apoptosis. Briefly, the cells were washed twice with cold PBS and then resuspended in 1× binding buffer (10 mM HEPES/NaOH, pH 7.4, 140 mM NaCl, 2.5 mM CaCl₂; 4° C.) at a concentration of about 1×10⁶ cells/mL. A 100-μL aliquot of cell suspension was transferred to 5-mL culture tubes, and 2.5 μL of Annexin V-FITC and 2.5-μL of propidium iodide (PharMingen) were added to each tube. The cell suspensions were gently mixed and incubated for 15 min at room temperature in the dark, after which 400 μL of binding buffer was added to each tube of cells. The cells were then washed once with 1× binding buffer. Analysis by flow cytometry was then conducted immediately, and no later than within one hour. The resulting analytic FACS graphs were typically divided into four quadrants wherein the upper right (UR) quadrant represented the number of necrotic cells; the lower left (LL) quadrant represented the number of viable cells; the lower right (LR) quadrant represented the number of apoptotic cells; and the upper left (UL) represented cells of an intermediate category, which generally represents cell debris and DNA fragments. The percentage of gated events in each quadrant was used to determine the total percentage of apoptotic and necrotic cells in each cell suspension.

Protein Tyrosine Kinase (PTK) Assay. A PTK assay kit (The Oncogene Research Products, Boston, Mass.) was used to determine the presence and relative amounts of protein kinase activity in tissue cytosols and cell extracts. Briefly, 1×10⁴ RG2 cells seeded in a 50-mL culture flask, and allowed to achieve confluency to form a monolayer at 37° C. in a CO₂ incubator. Protein tyrosine kinase was extracted from RG2 cells as suggested by the kit's manufacturer.

Briefly, RG2 cells were harvested after trypsinization, and cells were pelleted by centrifugation. Cells were resuspended with extraction buffer (20 mM Tris, pH 7.4, 50 mM NaCl, 1 mM EDTA, 1 mM EGTA, 0.2 mM PMSF, 1 μg/mL pepstatin, 0.5 μg/mL leupetin, 0.2 mM Na₃VO₄, 5 mM mercaptoethanol, 0.1% Triton X-100), and cells were lysed using a cell homogenizer. After centrifugation, supernatant was separated from the pellet for PTK assay. The supernatant was treated with superoxide dismutase. Pervanadate (100 mM) was used in the PTK assay to inhibit ATPase activity, and protein concentration was determined by Lowry's method using a Bio-Rad protein assay kit. Supernatant containing 50 μg protein was incubated, either with or without NS-1619 or valinomycin at various concentrations (1-50 μM), at room temperature.

Assay for PTK activity in RG2 cell-lysates was then performed according to the protocol supplied by the PTK assay kit's manufacturer. After adding the stop solution, absorbance measured in each well at 450 nm using a Spectramax Plus (Molecular Devices, Sunnyvale, Calif.).

In vivo studies in implanted tumors, A rat tumor model, which consisted of intracranially implanted RG2 cells (rat glioma cell line) in Wistar rats, was employed to investigate whether any of several potassium channel activators induce selective apoptosis of tumor cells in vivo.

The techniques for RG2 cell propagation and maintenance in tissue culture have been described (Sugita, M. and Black, K. L., Cyclic GMP-specific phosphodiesterase inhibition and intracarotid bradykinin infusion enhances permeability into brain tumors, Cancer Res. 58(5):914-20 [1998]; Inamura et al. [1994]; Nakano, S. et al., Increased brain tumor microvessel permeability after intracarotid bradykinin infusion is mediated by nitric oxide, Cancer Res. 56(17):4027-31 [1996]). Briefly, RG2 cells derived from a rat glioma are kept frozen until use, then are thawed and maintained in a monolayer culture in F12 medium with 10% calf serum.

The Wistar rats (approximately 140-160 g body weight) were anesthetized with intra-peritoneal ketamine (50 mg/kg), and glial cells (1×10⁵) were implanted into the right hemisphere, but not the contralateral hemisphere, by intracerebral injection suspended in 5 μL F12 medium (1-2% methylcellulose) by a Hamilton syringe. The implantation coordinates were 3-mm lateral to the bregma and 4.5 mm deep to the dural surface.

On the seventh day after tumor implantation, rats were anesthetized, the internal carotid artery was cannulated with P-50 polyethylene catheter, and the catheter was exteriorized so that the catheter was placed on the back of the rat for multiple infusions. Two rats were used for this study. One rat was given two doses of NS-1619 (20 μg/kg) on days 7 and 9, or 5 μM staurosporine on day 9, while another rat was administered vehicle (PBS, pH 7.4+1% v/v ethanol) as a control.

Ten days after RG2 implantation, the rats were euthanized under anesthesia and tumor and contralateral tumor tissues were carefully dissected for preparation of a single-cell suspension. Tissues were gently minced with a sharp surgical blade, using 18 G and 22 G needles; a single cell suspension was prepared by repeated aspirations with a syringe. Cells then were centrifuged at 1000 rpm for 5 minutes, and the supernatant was discarded. The resulting pellet was resuspended with 1-2 mL of PBS, and was washed twice in a similar manner. The final pellet of cells was resuspended in 100 μL 1× binding buffer as described hereinabove (supplied by PharMingen), was mixed well with 2.5 μL each of propidium iodide and FITC-conjugated Annexin V antibody, and was then incubated for 15 minutes in darkness. Finally, 400 μL of 1× binding buffer was added to the cell suspension and FACS analysis was performed as described hereinabove.

Immunohistochemical detection of K_{Ca} channels. Brain sections (12 μm thick) obtained from the permeability studies were incubated with 1:100 dilution of affinity-purified K_{Ca} channel antibody (Alomone Labs, Jerusalem, Israel) for 1 hour, and biotinylated horse anti-mouse immunoglobin (Vector Laboratories, Burlingame, Calif.) for 30 minutes. After washing 3 times with PBS, the peroxidase sites were visualized using an avidin:biotinylated enzyme complex (ABC) kit.

### Example 2

### Results

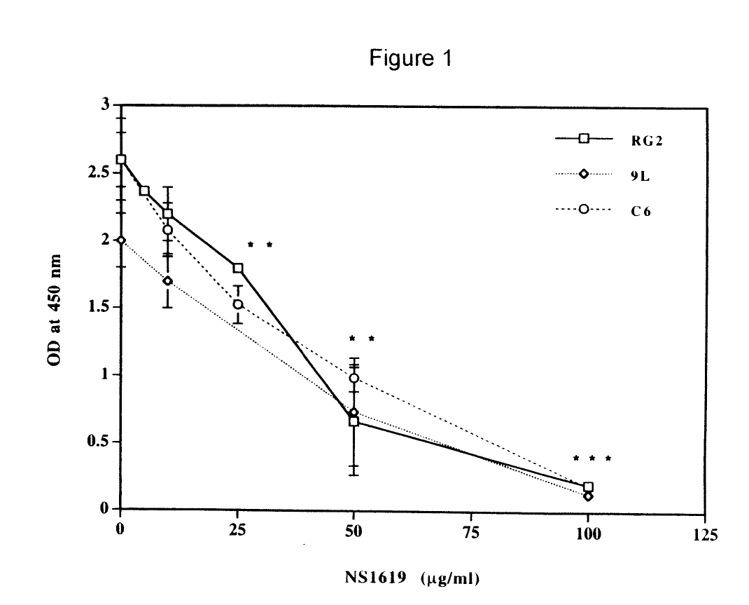

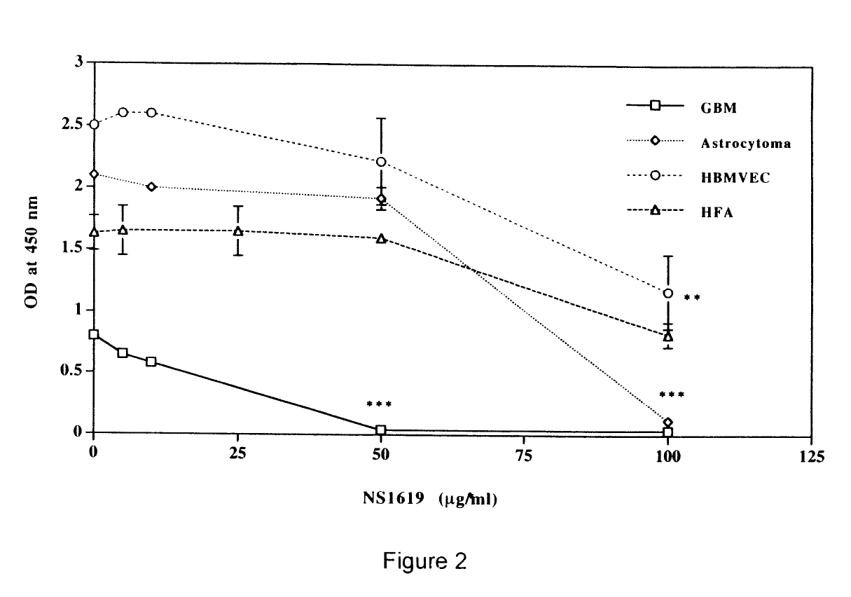

Cell proliferation inhibited by K_{Ca} activator. Cell proliferation assays were conducted, in vitro, as described herein, with rat glioma cells (RG2, C6 and 9L) or human normal cell lines, brain microvessel endothelial cells (HBMVEC), or fetal astrocytes (HFA). Results showed that K_{Ca} activator (NS-1619) significantly blocks cell proliferation selectively in the rat glioma cell lines in a dose-dependent manner (FIG. 1). Similarly, NS-1619 significantly blocked cell proliferation selectively in primary human glioma cell lines (GBM and astrocytoma) in a dose-dependent manner without affecting the normal HBMVEC and HFA cell lines (FIG. 2). All of the rat and human malignant cells studied, as well as normal cells, were insensitive to K_{ATP} channel agonist, minoxidil sulfate (data not shown).

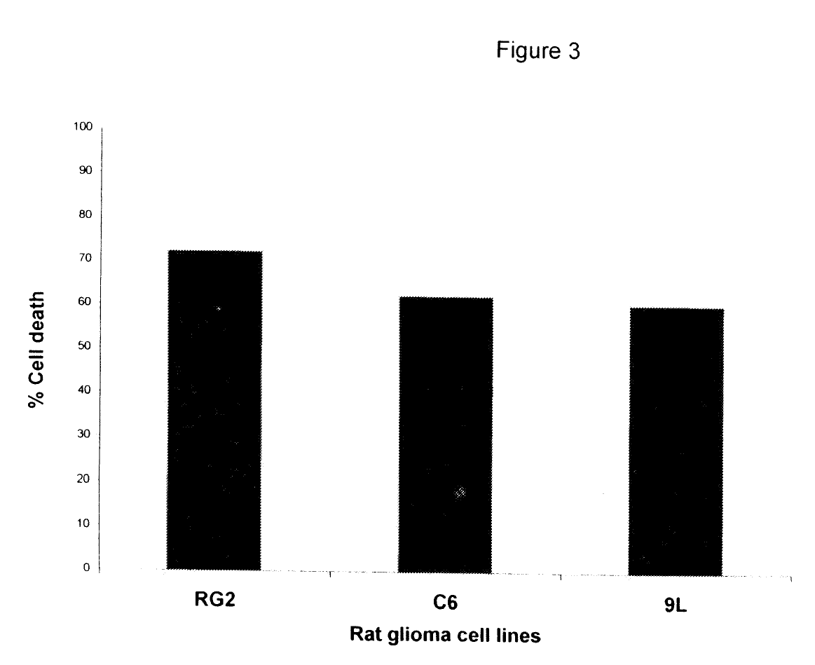

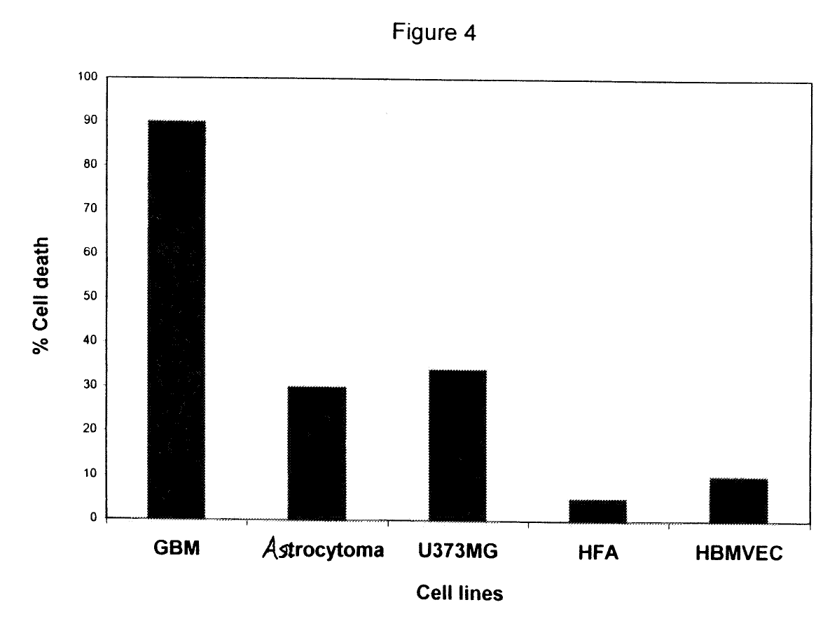

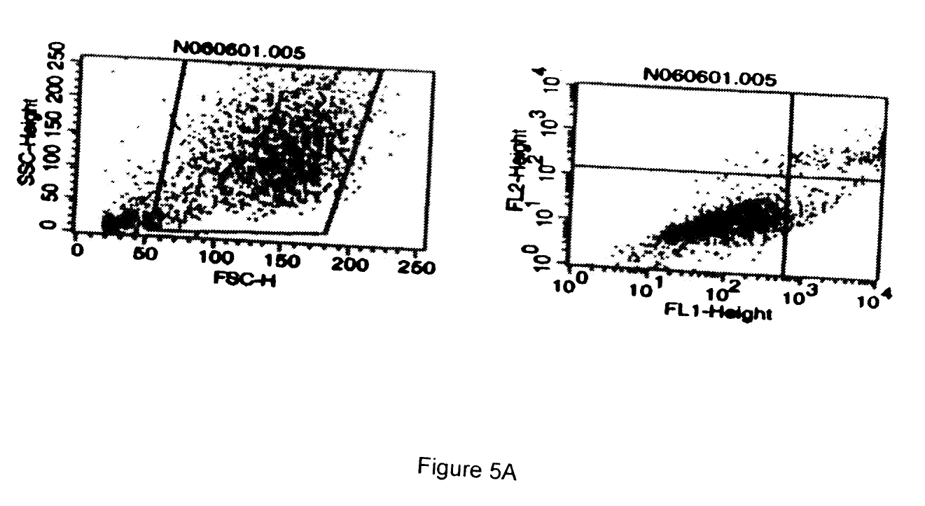

FIG. 3 illustrates that a differential sensitivity to NS-1619 (50 μg/mL) that was observed among rat glioma cell lines RG2, C6, and 9L, with RG2 being most sensitive (70% cell death). A similar differential sensitivity to NS-1619 at 50 μg/mL was observed with primary human GBM, astrocytoma, and U373MG glioma cell lines, GBM being most sensitive (90% cell death; FIG. 4). Normal cell lines HBMVEC and HFA were insensitive to NS-1619 at 50 μg/mL concentration (FIG. 4).

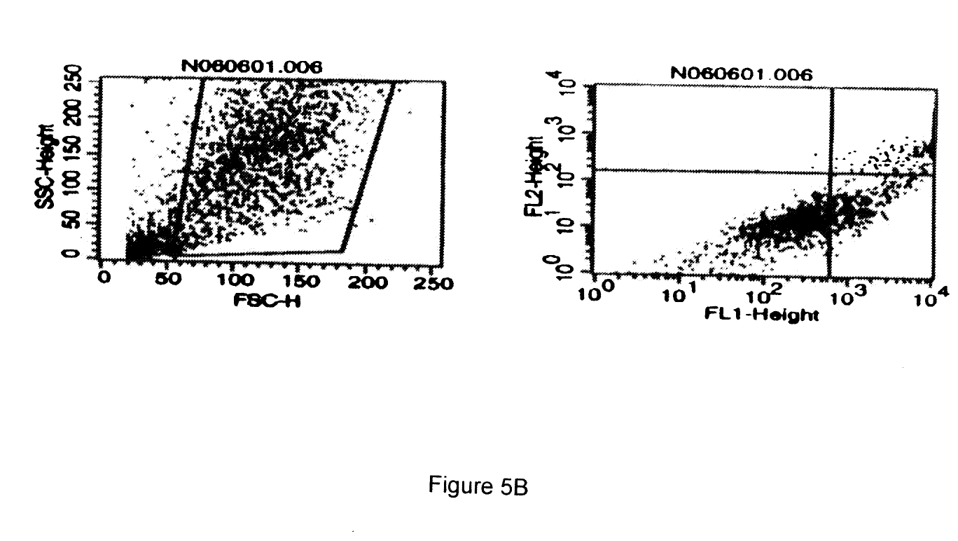

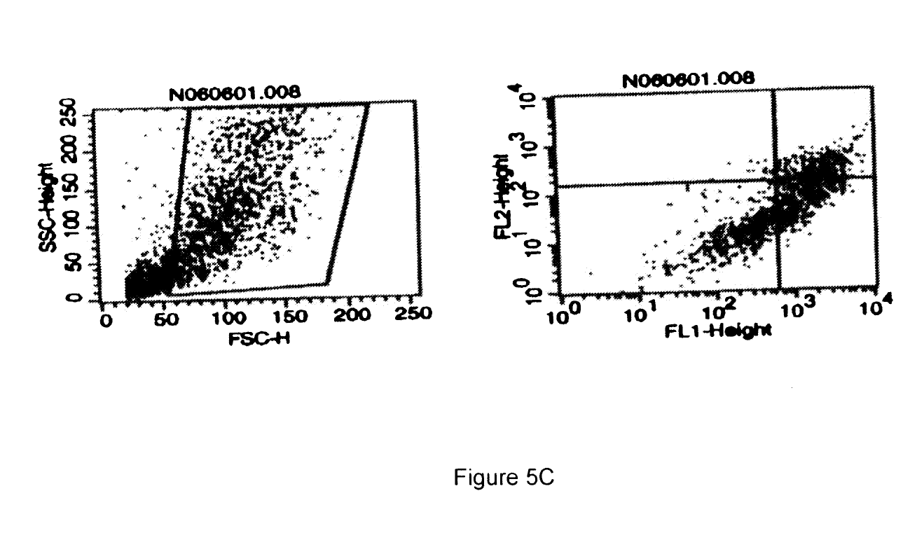

Apoptosis induction by K_{Ca} activator. FACS analysis showed that administration of NS-1619 (50 μg/mL) to U87 cells resulted in 21% apoptotic cells and 14% necrotic cells (FIG. 5A), compared to a vehicle (negative control) that had 5% apoptotic and 7% necrotic cells (FIG. 5B). U87 cells exposed to 5 μM staurosporin (positive control), a known inducer of apoptosis, showed 31% apoptotic cells and 27% necrotic cells (FIG. 5C).

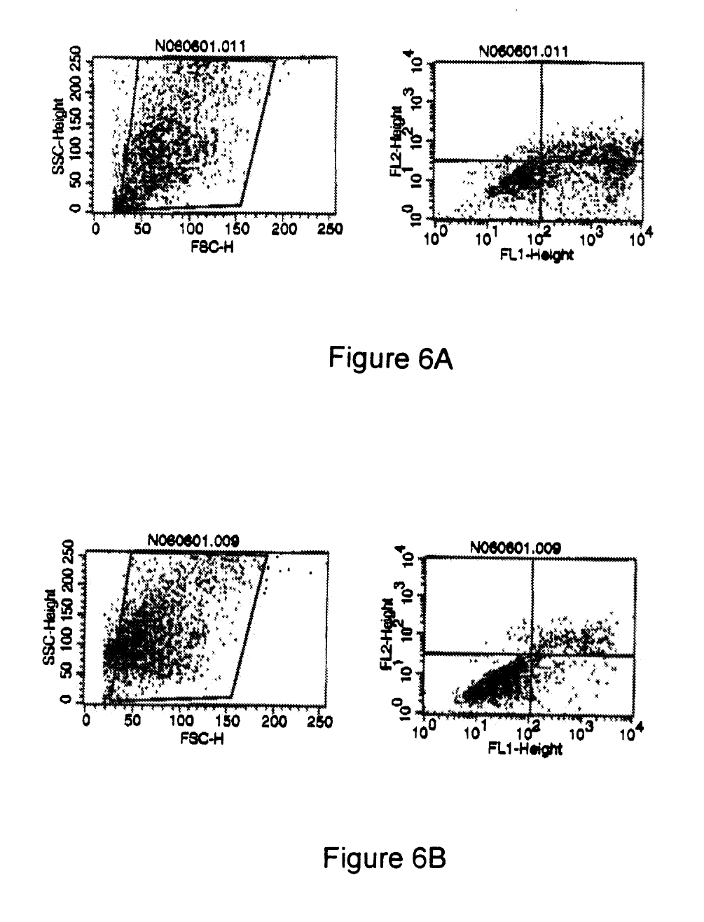

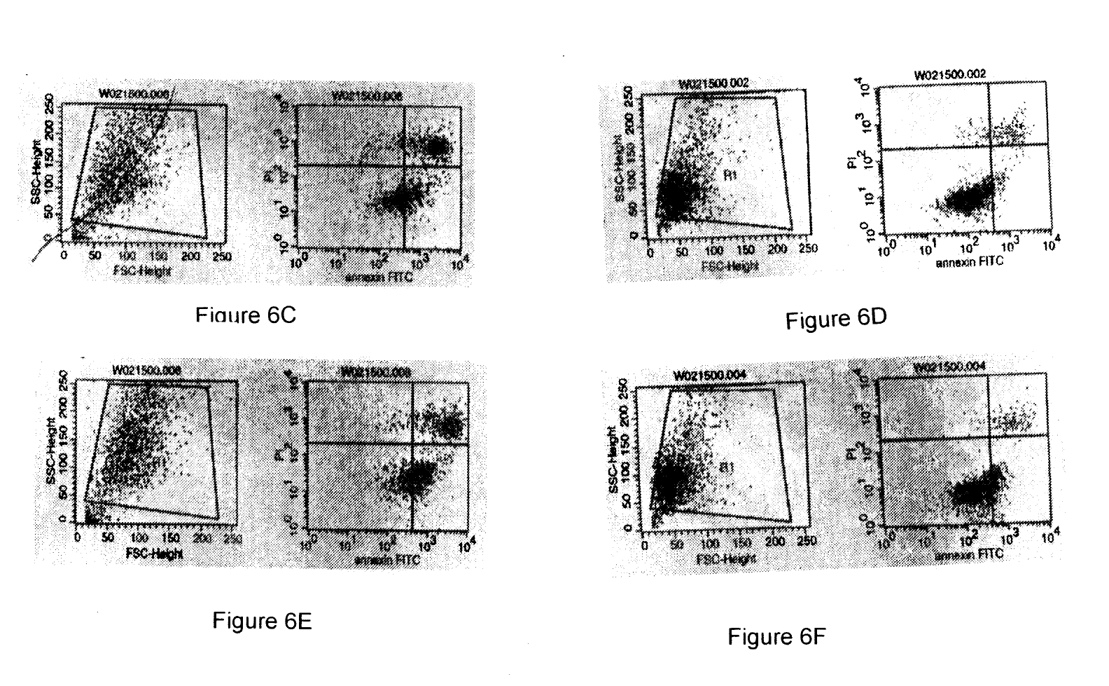

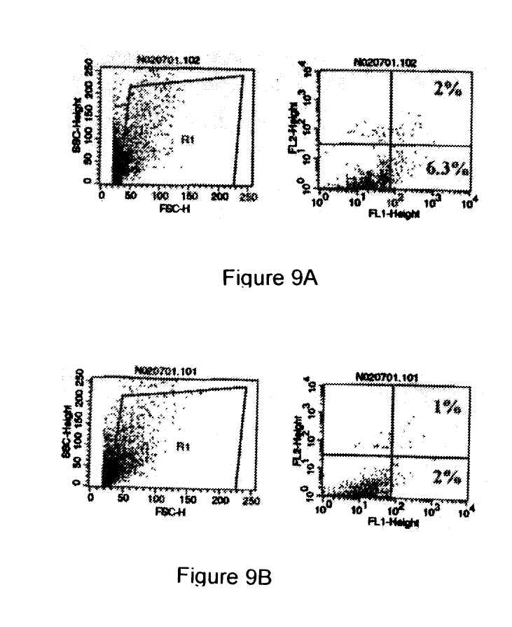

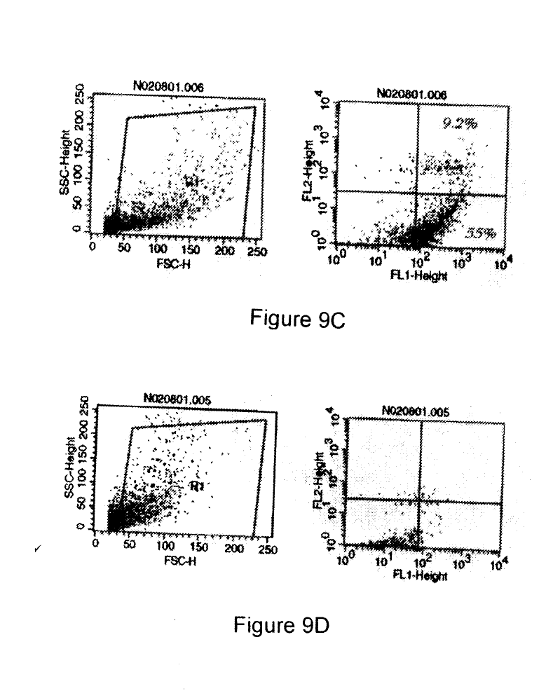

Similarly, FACS analysis showed that administration of NS-1619 (50 μg mL) to malignant RG2 cells greatly increased apoptosis (29%) and necrosis (29%) (FIG. 6A), compared to the percentage of apoptotic (5%) and necrotic (11%) cells in untreated RG2 cell suspensions (FIG. 6B). Similar results were obtained with NS-1619-treated malignant C6 cells (25% apoptotic, 35% necrotic C6 cells; FIG. 6C) and 9L cells (42% apoptotic, 32% necrotic 9L cells; FIG. 6E), compared to 5% apoptotic and 10% necrotic among untreated C6 cells (FIG. 6D), and 15% apoptotic and 7% necrotic among untreated 9L cells (FIG. 6F).

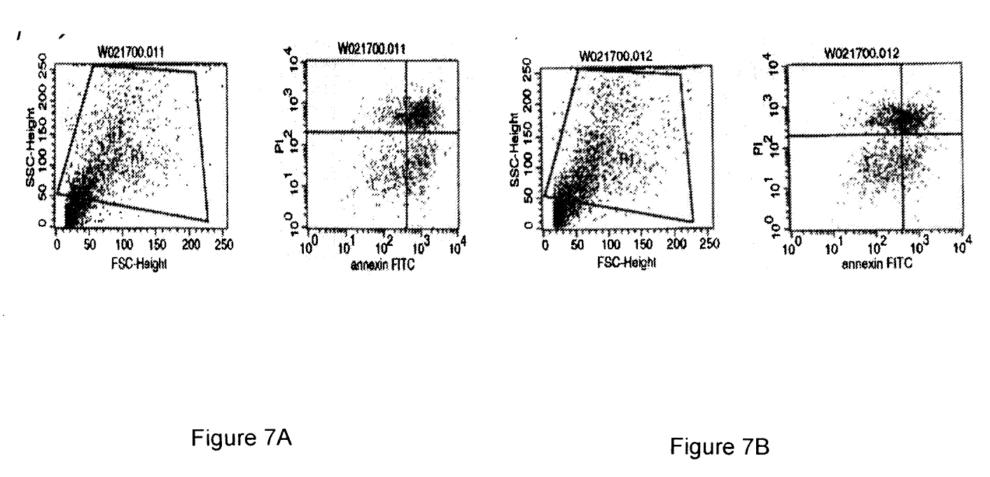

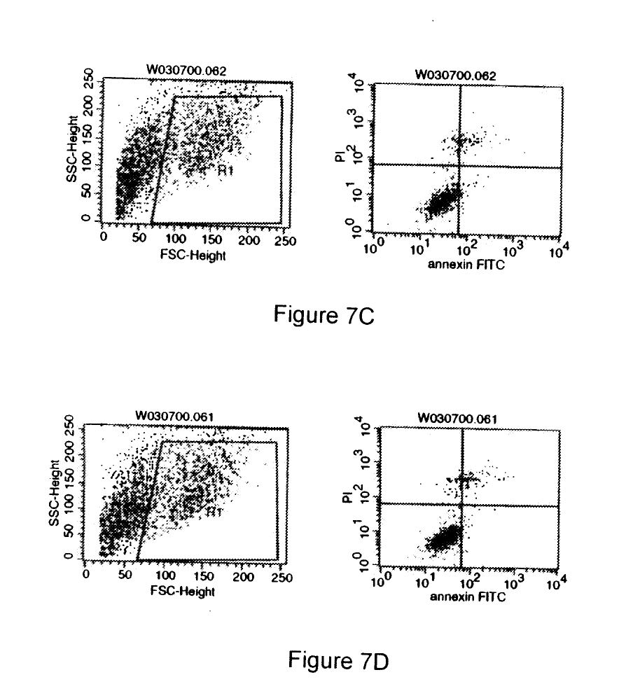

FACS analysis shows that NS-1619 (50 μg/mL) treatment greatly increased apoptosis (24%) and necrosis (49%) among primary human GBM cells (FIG. 7A), compared to 12% apoptotic and 36% necrotic cells among untreated GBM primary cells (FIG. 7B). However, NS-1619 (50 μg/mL) treatment did not significantly induce either apoptosis or necrosis in non-malignant HBMVEC (4% (apoptosis in both treated and vehicle controls; FIG. 7C and FIG. 7D).

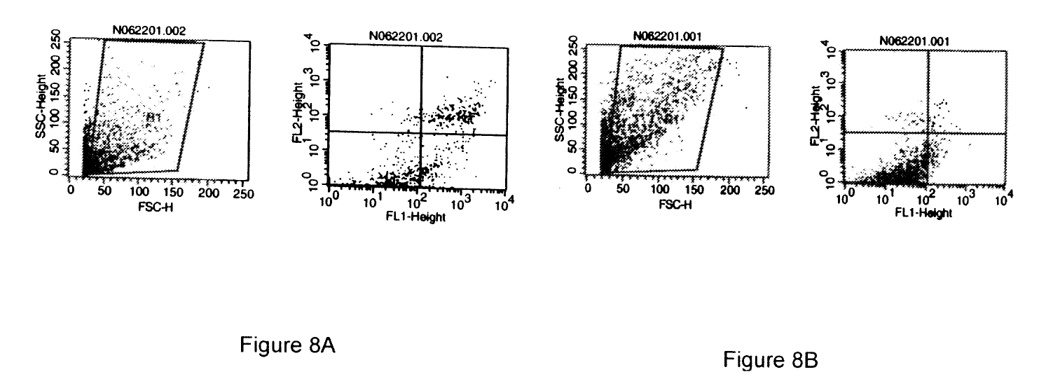

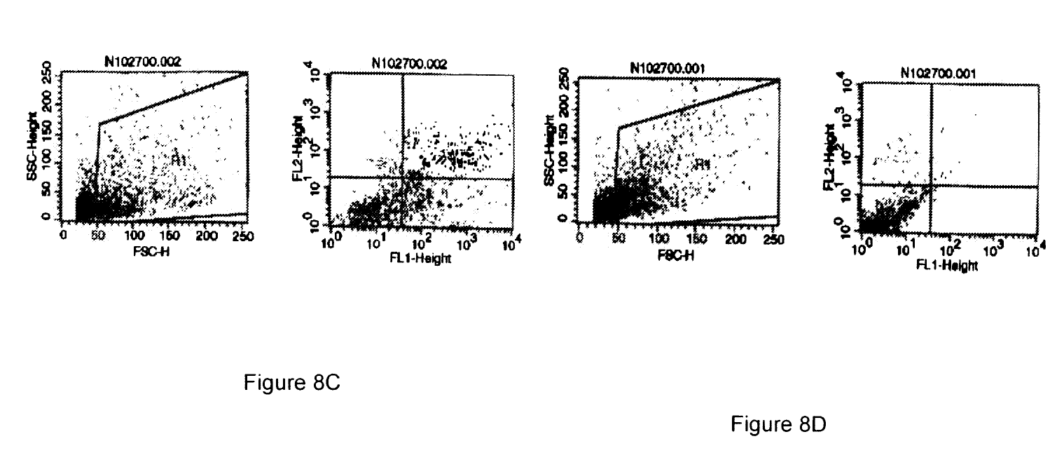

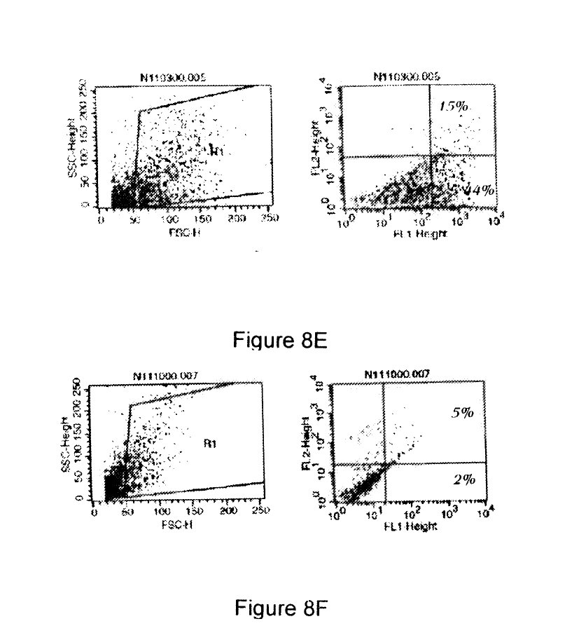

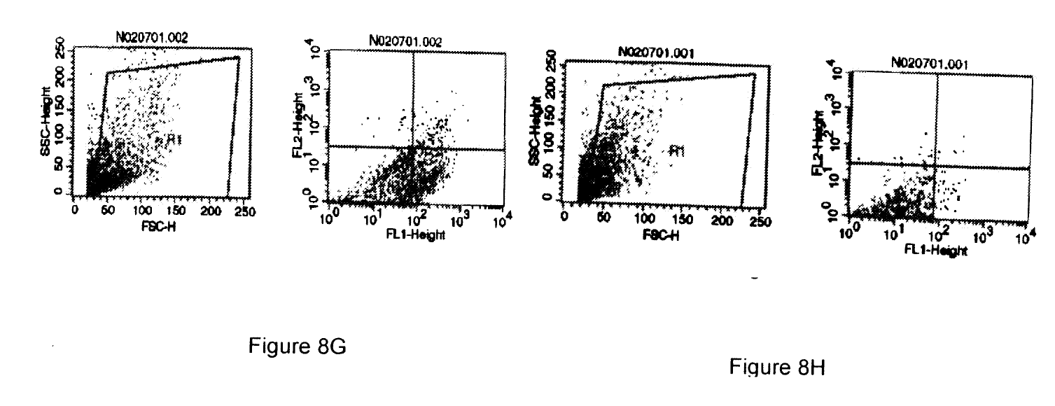

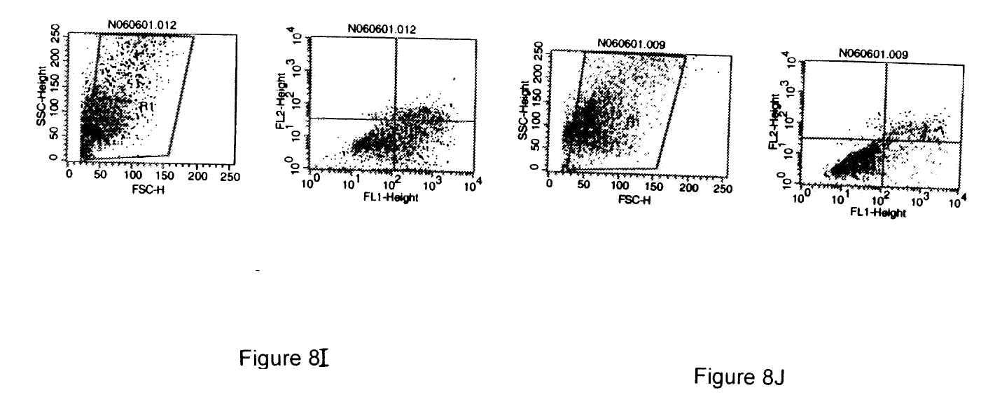

Intracarotid bolus infusion of a phosphate buffered saline vehicle only (PBS, pH 7.4, 0.5% v/v ethanol), i.e., minus K_{Ca} activator, on two consecutive days to rats with implanted RG2 glioma tumors, the tumor tissue contained 12% apoptotic cells and 18% necrotic (FIG. 8A). The contralateral normal brain tissue from the same rat contained a negligible number of apoptotic cells after PBS vehicle infusion (5% apoptotic cells, 2% necrotic cells; FIG. 5B). In contrast, a rat with implanted RG2 tumors that was given NS-1619 treatment (two consecutive daily doses of 50 or 100 μg NS-1619/kg body mass). A rat receiveing 50 μg NS-1619/kg body mass had 28% apoptotic and 24% necrotic cells with the RG2 tumor (FIG. 5C) and 1% apoptotic and 1% necrotic cells in contralateral normal brain tissue (FIG. 8D). One rat receiving 100 μg NS-1619/kg body mass/day for two days had 44% apoptosis and 15% necrosis within the RG2 tumor (FIG. 5E), compared to negligible apoptosis in the normal contralateral brain tissue of the same rat (2% apoptotic cells, 5% necrotic; FIG. 8F). Another rat receiving 100 μg NS-1619/kg body mass/day for two days had 34% apoptosis and 9% necrosis within the RG2 tumor (FIG. 8G), compared to negligible apoptosis in the normal contralateral brain tissue of the same rat (2% apoptotic cells, 1% necrotic; FIG. 5H). A representative positive control, in which the known apoptotic agent staurosporin (20 μg/kg body mass) was injected as a bolus, instead of NS-1619, resulted in 35% apoptotic cells and 17% necrotic cells within the RG2 tumor tissue (FIG. 8I), compared to 5% apoptotic cells and 11% necrotic cells in normal contralateral brain tissue (FIG. 8J).

When the treatment period was extended to three consecutive days the induction of apoptosis by K_{Ca} channel activator was even more pronounced. Intracarotid bolus infusion of a phosphate buffered saline vehicle only (PBS, pH 7.4, 1% v/v ethanol), i.e., minus K_{Ca} channel activator, on three consecutive days to rats with implanted RG2 glioma tumors, the tumor tissue contained 6.3% apoptotic cells and 2% necrotic cells (FIG. 9A). The contralateral normal brain tissue contained a negligible number of apoptotic cells after PBS vehicle infusion (2% apoptotic cells, 1% necrotic cells; FIG. 9B). In a rat that received three consecutive daily bolus doses of NS-1619 (100 μg/kg body mass), an even higher degree of apoptosis (55%) and necrosis (9%) was detected in implanted RG2 tumor tissue (FIG. 9C), compared with contralateral normal brain (2% apoptotic and 1% necrotic cells; FIG. 9D).

Together these data also show that infusion of NS-1619 for two or three consecutive days at up to 100 μg/kg body mass didn't affect normal brain tissue considerably, while inducing a selective cell death in tumor tissue.

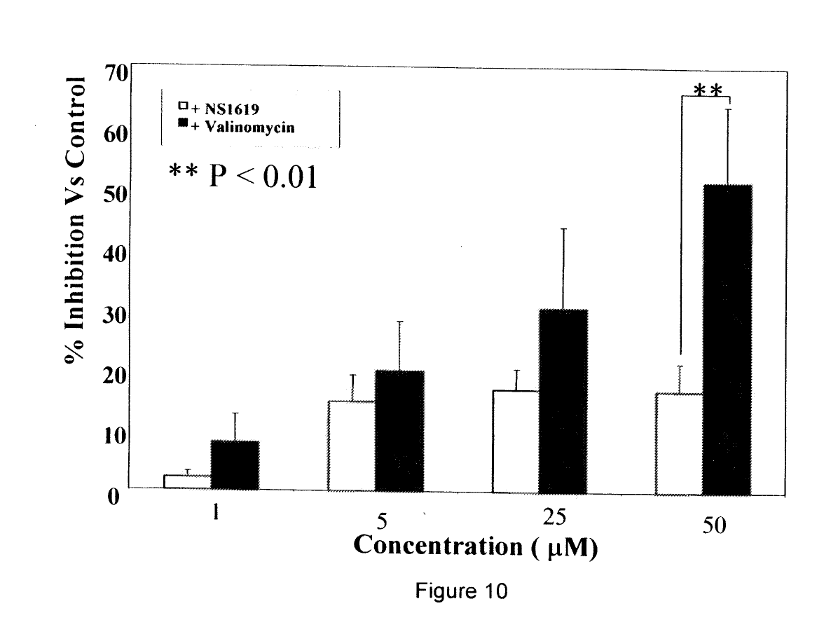

Experiments showed that induction of apoptosis by the K_{Ca} channel activator did not likely operate by disruption of a protein tyrosine kinase-mediated pathway. Valinomycin (a K⁺ ionophore) inhibited PTK activity in RG2 cell lysate in a dose-dependent manner, while NS-1619 did not significantly affect PTK activity in RG2 cell lysate (FIG. 10).

Immunohistochemical Analysis Shows Potassium Channels are More Abundant in Neovasculature and Malignant Cells Compared to Normal Tissue.

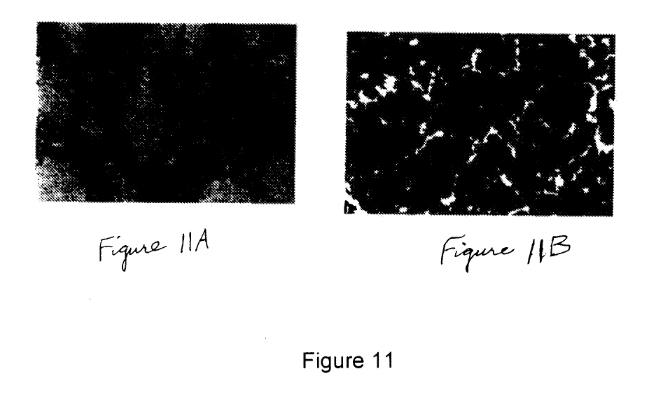

H_{Ca} channel protein was immunolocalized using a specific antibody as described above. Immunohistochemical analysis showed that K_{Ca} channels were more highly localized in tumor tissue in RG2 bearing rat brain sections (FIG. 11B), compared to sections of normal contralateral tissue (FIG. 11A). These immunohistochemical results are consistent with results showing activation of K_{Ca} channels by NS-1619 selectively induced apoptosis in malignant cells compared to normal cells.

Together, the apoptosis and immunohistochemical data demonstrate that compounds that activate calcium dependent potassium channels can be used to selectively induce apoptosis of malignant cells in malignant tumor tissue.

The foregoing examples being illustrative but not an exhaustive description of the embodiments of the present invention, the following claims are presented.

1-45. (canceled)

46. A kit for selectively inducing apoptosis of malignant cells, comprising:
a pharmaceutically acceptable formulation of a calcium-activated potassium channel activator; and
instructions for using it to selectively induce apoptosis of malignant cells.

47. The kit of claim 46, wherein the calcium-activated potassium channel activator is NS-1619.

48. The kit of claim 46, wherein the calcium-activated potassium channel activator is triethylamine.

49. The kit of claim 46, wherein the calcium-activated potassium channel activator is bradykinin or a bradykinin analog.

50. The kit of claim 46, wherein the calcium-activated potassium channel activator is a soluble guanylyl cyclase activator, YC-1, or a guanylyl cyclase activating protein.
